(19) United States
(12) Patent Application Publication
(10) Pub. No.: US 2024/0225641 A1
(43) Pub. Date: Jul. 11, 2024

(54) SURGICAL STAPLING DEVICE WITH FORCE REDUCTION MECHANISM

(71) Applicant: Covidien LP, Mansfield, MA (US)

(72) Inventors: Zhaokai Wang, Shanghai (CN); Xiliang Zhang, Shanghai (CN)

(21) Appl. No.: 18/558,757

(22) PCT Filed: May 6, 2021
(86) PCT No.: PCT/CN2021/091901

§ 371 Date: Nov. 3, 2023

(51) Int. Cl. A61B 17/072; A61B 17/00

(52) U.S. Cl. CPC A61B 17/072; A61B 2017/00367; A61B 2017/07285

(57) ABSTRACT

A manually operated surgical stapling device includes an end effector and a firing mechanism that is configured to reduce the torque required to fire staples from the end effector of the stapling device. The firing mechanism includes a trigger, a thrust bar, and a firing link that is secured to the trigger and engaged with the thrust bar. The trigger is coupled to a handle assembly of the stapling device and to the firing link to minimize the torque required to move the trigger between unactuated and actuated positions.

### FIELD

This technology is generally related to surgical stapling devices and, more particularly, to a manually actuated surgical stapling device with a firing force reduction mechanism.

### BACKGROUND

Surgical stapling devices are commonly used during a variety of surgical procedures to expedite dissection and suturing of tissue and minimize trauma to a patient. These devices can be manually operated or powered, e.g., motorized, and are available in a variety of configurations including linear stapling devices, circular stapling devices, and transverse stapling devices. Transverse stapling devices include an end effector that has an anvil assembly and a cartridge assembly that have axes that are transverse to a longitudinal axis of the surgical stapling device. The cartridge supports a plurality of staples that are simultaneously advanced into the anvil assembly when the stapling device is fired to form staples in tissue.

Manually actuated stapling devices include a trigger that is engaged with a thrust bar via a link such that operation of the trigger advances the thrust bar within the stapling device. The thrust bar is operatively engaged with a pusher that is supported within a staple cartridge of the cartridge assembly. When the thrust bar is advanced, the pusher is advanced within the staple cartridge to simultaneously eject the staples from the cartridge assembly into the anvil assembly. Typically, these stapling devices also include a knife assembly that is engaged with the pusher and the thrust bar and is advanced upon operation of the trigger to simultaneously cut tissue clamped between the anvil and cartridge assemblies as the staples are ejected into the anvil assembly. The simultaneous stapling and cutting of tissue requires a substantial driving torque to be applied to the trigger by a clinician to actuate the stapling device.

A continuing need exists in the art for a surgical stapling device that includes a mechanism for minimizing the driving torque required to actuate the stapling device.

### SUMMARY

This disclosure is directed to a manually operated surgical stapling device that includes an end effector and a firing mechanism to reduce the torque required to actuate a trigger to fire staples and cut tissue.

Aspects of this disclosure are directed to a surgical stapling device that includes a handle assembly, an elongate body, and an end effector. The handle assembly includes a housing, a firing link, and a trigger. The housing defines a stationary handle. The trigger is mounted to the housing by a first pivot member and has a first portion defining a grip and a second portion having a second end spaced from the first portion. The firing link defines a longitudinal axis “Y” and has a first end portion and a second end portion. The first end portion of the firing link is coupled to the second portion of the trigger by a second pivot member. The trigger is pivotable about the first pivot member towards the stationary handle through a firing stroke from an unactuated position to an actuated position. An axis “Z” extends through the first and second pivot members. The elongate body extends distally from the handle assembly and includes a frame and a thrust bar. The thrust bar is movably supported on the frame between retracted and advanced positions. The frame has a distal portion and a proximal portion. The thrust bar defines a longitudinal axis “X” and has a distal portion and a proximal portion. The proximal portion of the thrust bar is engaged with the second end portion of the firing link such that movement of the trigger through the firing stroke moves the thrust bar from the retracted position to the advanced position. The end effector is supported on the distal portion of the frame of the elongate body and includes an end effector frame, an anvil assembly, and a cartridge assembly. The end effector frame supports the anvil assembly and the cartridge assembly. The cartridge assembly includes a cartridge body, a knife assembly, and a pusher. The knife assembly is engaged with the distal portion of the thrust bar such that movement of the thrust bar from its retracted position to its advanced position moves the knife assembly and the pusher within the cartridge body from retracted positions to advanced positions. The axes “Y” and “Z” define an angle 3, wherein the angle β is from about 95 degrees to about 110 degrees when the trigger is in the unactuated position and from about 155 degrees to about 170 degrees when the trigger is in the actuated position.

Other aspects of the disclosure are directed to a handle assembly that includes a housing, a firing link, a trigger, and a thrust bar. The housing defines a stationary handle. The trigger is mounted to the housing by a first pivot member and has a first portion defining a grip and a second portion having a second end spaced from the first portion. The firing link defines a longitudinal axis “Y” and has a first end portion and a second end portion. The first end portion of the firing link is coupled to the second portion of the trigger by a second pivot member. The trigger is pivotable about the first pivot member towards the stationary handle through a firing stroke from an unactuated position to an actuated position. The thrust bar extends from a distal portion of the housing and defines a longitudinal axis “X”. An axis “Z” extends through the first and second pivot members. The axes “Y” and “Z” define an angle β, wherein the angle β is from about 95 degrees to about 110 degrees when the trigger is in the unactuated position and from about 155 degrees to about 170 degrees when the trigger is in the actuated position.

Other aspects of this disclosure are directed to a surgical stapling device that includes a handle assembly, an elongate body, and an end effector. The handle assembly includes a housing, a firing link, and a trigger. The housing defines a stationary handle. The trigger is mounted to the housing by a first pivot member and has a first portion defining a grip and a second portion having a second end spaced from the first portion. The firing link defines a longitudinal axis “Y” and has a first end portion and a second end portion. The first end portion of the firing link is coupled to the second portion of the trigger by a second pivot member. The trigger is pivotable about the first pivot member towards the stationary handle through a firing stroke from an unactuated position to an actuated position. An axis “Z” extends through the first and second pivot members. The elongate body extends distally from the handle assembly and includes a frame and a thrust bar. The thrust bar is movably supported on the frame between retracted and advanced positions. The frame has a distal portion and a proximal portion. The thrust bar defines a longitudinal axis “X” and has a distal portion and a proximal portion. The proximal portion of the thrust bar is engaged with the second end portion of the firing link such that movement of the trigger through the firing stroke moves the thrust bar from the retracted position to the advanced position. The end effector is supported on the distal portion of the frame of the elongate body and includes an end effector frame, an anvil assembly, and a cartridge assembly. The end effector frame supports the anvil assembly and the cartridge assembly. The cartridge assembly includes a cartridge body, a knife assembly, and a pusher. The knife assembly is engaged with the distal portion of the thrust bar such that movement of the thrust bar from its retracted position to its advanced position moves the knife assembly and the pusher within the cartridge body from retracted positions to advanced positions. The axes “X” and “Y” define an angle α, wherein the angle α, when the trigger is in the unactuated position, is greater the angle α when the trigger is in the actuated position.

In some aspects of the disclosure, the end effector frame includes first and second transverse portions and a longitudinal portion that interconnects the first and second transverse portions.

In certain aspects of the disclosure, the first and second transverse portions define a gap.

In aspects of the disclosure, the anvil assembly is supported on the first transverse portion and the cartridge assembly is supported adjacent the second transverse portion.

In some aspects of the disclosure, the stapling device includes a clamp slide assembly having a distal portion supported within the gap.

In certain aspects of the disclosure, the clamp slide assembly is movable between retracted and advanced positions to move the cartridge assembly in relation to the anvil assembly between open and clamped positions.

In aspects of the disclosure, the proximal portion of the thrust bar defines a recess and the distal portion of the firing link is received within the recess.

In some aspects of the disclosure, the first end portion of the firing link includes a C-shaped clip that receives the second pivot member.

In certain aspects of the disclosure, the first pivot member is positioned between the second pivot member and the grip of the trigger.

In aspects of the disclosure, the first pivot member is positioned proximally of the second pivot member when the trigger is in the unactuated position.

Other features of the disclosure will be appreciated from the following description.

### BRIEF DESCRIPTION OF THE DRAWINGS

Various aspects of the disclosure are described herein below with reference to the drawings, wherein:

FIG. 1 is a side view of a Prior Art stapling device illustrating a firing mechanism of the stapling device with the remaining components of the stapling device shown in phantom;

FIG. 2 is an enlarged, cutaway view of the firing mechanism of the stapling device shown in FIG. 1 in an unactuated position;

FIG. 3 is a side perspective view of a stapling device according to aspects of the disclosure illustrating a firing mechanism of the stapling device with the remaining components of the stapling device shown in phantom;

FIG. 4 is an exploded perspective view of the firing mechanism of the stapling device shown in FIG. 3;

FIG. 4A is an enlarged, cutaway view of the firing mechanism of the stapling device shown in FIG. 1 in an unactuated position;

FIG. 5 is a side view of the stapling device shown in FIG. 3 in a pre-fired state illustrating the firing mechanism of the stapling device with the remaining components of the stapling device shown in phantom;

FIG. 5A is an enlarged view of the end effector of the stapling device shown in FIG. 3 with the end effector in a clamped and fired state with the end effector frame and a cartridge body of a cartridge assembly of the end effector shown in phantom; and

FIG. 6 is a side view of the stapling device shown in FIG. 5 in a fired state illustrating the firing mechanism of the stapling device with the remaining components of the stapling device shown in phantom;

### DETAILED DESCRIPTION

The disclosed surgical stapling device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the stapling device in its customary manner, while the term “distal” is used generally to refer to that portion of the device that is farther from the clinician during use of the stapling device in its customary manner. In addition, the term “clinician” is used generally to refer to medical personnel including doctors, nurses, and support personnel. Further, directional terms such as front, rear, upper, lower, top, bottom, and similar terms are used to assist in understanding the description and are not intended to limit the disclosure.

This disclosure is directed to a manually operated surgical stapling device that includes an end effector and a firing mechanism to reduce the torque required to fire staples and cut tissue. The firing mechanism includes a trigger, a thrust bar, and a firing link that is secured to the trigger and engaged with the thrust bar. The trigger is coupled to a housing of a handle assembly of the stapling device about a first pivot member and coupled to a first end of the firing link about a second pivot member. A second end of the firing link is engaged with the thrust bar such that movement of the trigger about the first pivot member effects longitudinal movement of the thrust bar in relation to the end effector. The first and second pivot members are positioned on the trigger to minimize the torque required to move the trigger between unactuated and actuated positions.

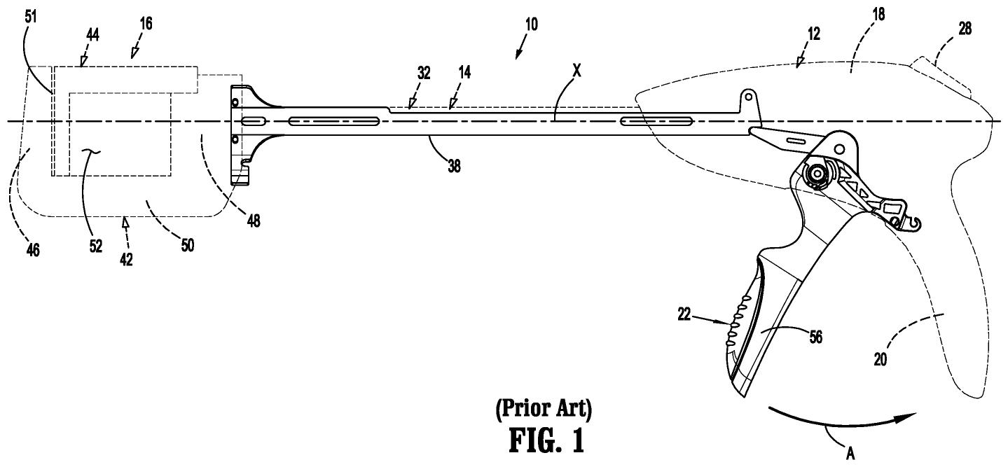

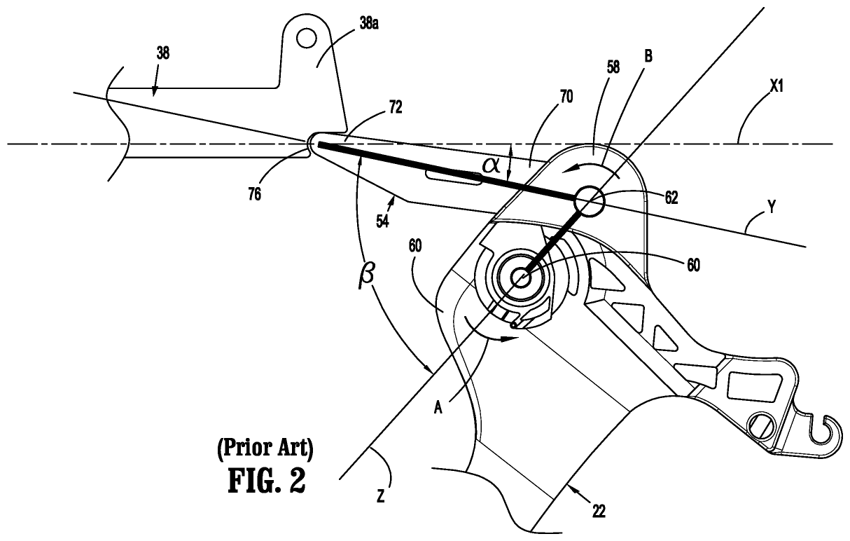

FIGS. 1 and 2 illustrate a “Prior Art” surgical stapling device according to aspects of the disclosure shown generally as stapling device 10. The stapling device 10 includes a handle assembly 12, an elongate body 14 that extends distally from the handle assembly 12, and an end effector 16 that is supported on a distal portion of the elongate body 14. The elongate body 14 defines a longitudinal axis “X”. The handle assembly 12 includes a housing 18 that defines a stationary handle 20 and supports a movable trigger 22. In aspects of the disclosure, the trigger 22 is supported by the housing 18 to pivot towards the stationary handle 20 in the direction of arrow “A” between non-actuated and actuated positions to actuate the end effector 16, i.e., move the end effector 16 between open and clamped positions and fire the stapling device 10. The handle assembly 12 includes a release button 28 that can be depressed to move the end effector 16 from the clamped position to the open position. For a more detailed description of a representative handle assembly 12, see, e.g., U.S. Pat. No. 6,817,508 (“the '508 patent”).

The stapling device 10 includes a frame 32 that extends from the handle assembly 12 and includes a distal portion that supports the end effector 16. The frame 32 includes spaced frame members that define a channel that receives a thrust bar 38 and the alignment pin pusher (not shown). The thrust bar 38 is movable within the channel of the frame 32 between retracted and advanced positions in response to movement of the trigger 22 in the direction of arrow “A” to actuate the stapling device 10. The channel defined by the frame 32 also receives a clamp slide assembly (not shown) that extends into the end effector 16 as described in further detail below. For a more detailed description of the interaction between the trigger 22, the thrust bar 38, and the clamp slide assembly, see the '508 patent.

The end effector 16 includes an end effector frame 42 and a cartridge assembly 44. The end effector frame 42 is secured to the distal portion of the frame 32 of the stapling device 10 and includes a first transverse portion 46, a second transverse portion 48, and a longitudinal portion 50 that interconnects the first transverse portion 46 and the second transverse portion 48. The first and second transverse portions 46, 48 are spaced from each other to define a gap 52 between the first and second transverse portions 46, 48. In some aspects of the disclosure, the first and second transverse portions 46, 48 are curved along axes transverse to the longitudinal axis “X” of the elongate body 14 of the stapling device 10. In some aspects of the disclosure, the first and second transverse portions 46, 48 are formed from a plurality of linear portions, e.g., three, that are positioned at angles in relation to each other to define a curved configuration. The first transverse portion 46 of the end effector frame 42 supports an anvil assembly 51.

FIG. 2 illustrates a central portion of a firing mechanism of the stapling device 10 (FIG. 1) which includes the thrust bar 38, the trigger 22, and a firing link 54. In aspects of the disclosure, the trigger 22 includes a first end portion 56 forming a grip (FIG. 1) and a second end portion 58. The second end portion 58 of the trigger 22 is pivotably secured to the housing 18 (FIG. 1) by a first pivot member 60 and pivotably secured to the firing link 54 by a second pivot member 62. The second pivot member 62 is positioned nearer an end of the trigger 22 opposite the first end portion 56 of the trigger 22 such that when the trigger 22 is moved in the direction of arrow “A” from the unactuated position (FIG. 1) towards the actuated position, the second pivot member 62 moves distally in the direction indicated by arrow “B” about the first pivot member 60.

The firing link 54 defines a longitudinal axis “Y” that extends through the second pivot member 62 and has a proximal portion 70 and a distal portion 72. The proximal portion 70 is pivotably secured to the second end portion 58 of the trigger 22 by the second pivot member 62. The distal portion 72 of the firing link 54 has a tapered configuration that is received in a recess 76 in the proximal end portion 38a of the thrust bar 38. An axis “Z” extends through the first and second pivot members 60, 62. The thrust bar 38 defines a longitudinal axis “X1” that is substantially parallel to the longitudinal axis “X” of the elongate body 14.

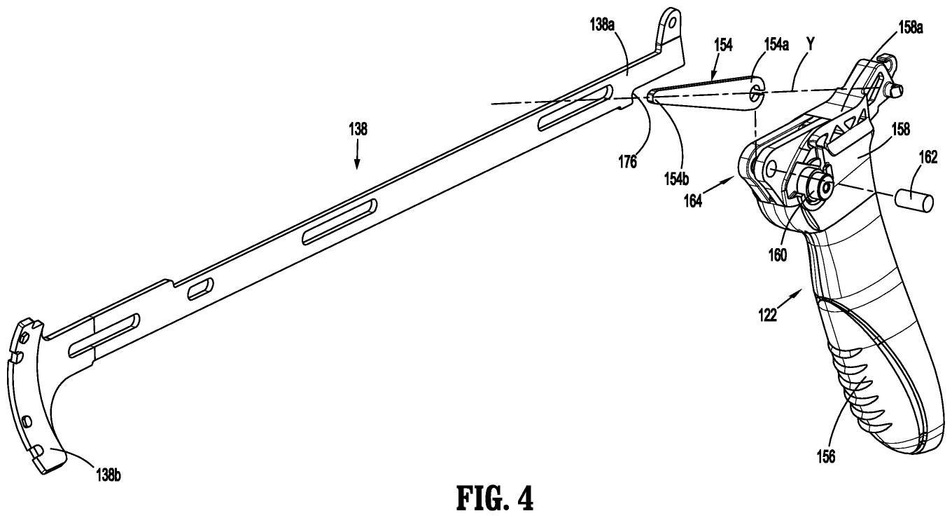

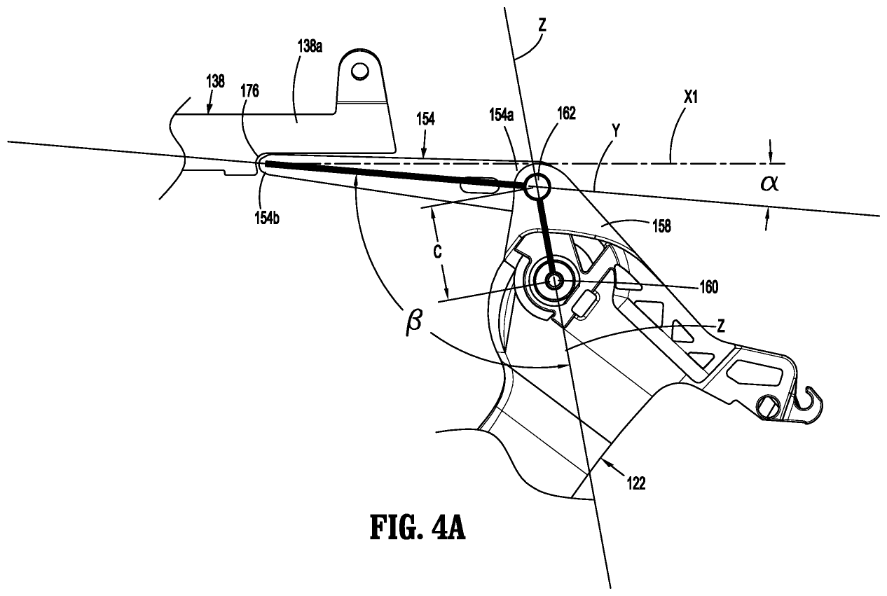

When the trigger 22 is in the unactuated position, the axes “X1” and “Y” define an angle α and the axes “Y” and “Z” define an angle β. In this position, the first pivot member 60 is positioned distally of the second pivot member 62. When tissue is clamped between the anvil assembly 51 and the cartridge assembly 44 and the stapling device 10 (FIG. 1) is fired by pivoting the trigger 22 in the direction indicated by arrow “A” in FIG. 1 from its unactuated position to its actuated position, the torque needed to pivot the trigger 22 to the actuated position (assuming a constant load) can be calculated using the following formula:

$$T = \frac{L \times c \times sin β}{cos α}$$

where T is the torque, L is the load, and c (FIG. 4A) is the distance between the first pivot member 160 and the second pivot member 162. When the trigger 22 is moved from its unactuated position towards its actuated position, α changes from about 15 degrees to about 11 degrees and β changes from about 65 degrees to about 106 degrees. As is evident from the formula, where the load L is constant, the torque T increases most notably as β approaches 90 degrees. When β is 90 degrees, mechanical advantage of the trigger 22 is at its minimum. In the prior art stapling device 10, β approaches 90 degrees at the end of the firing stroke. It is at this point that the load is at its maximum due to staple formation and cutting of tissue. This makes it difficult and tiresome for a clinician to move the trigger all the way through the firing stroke.

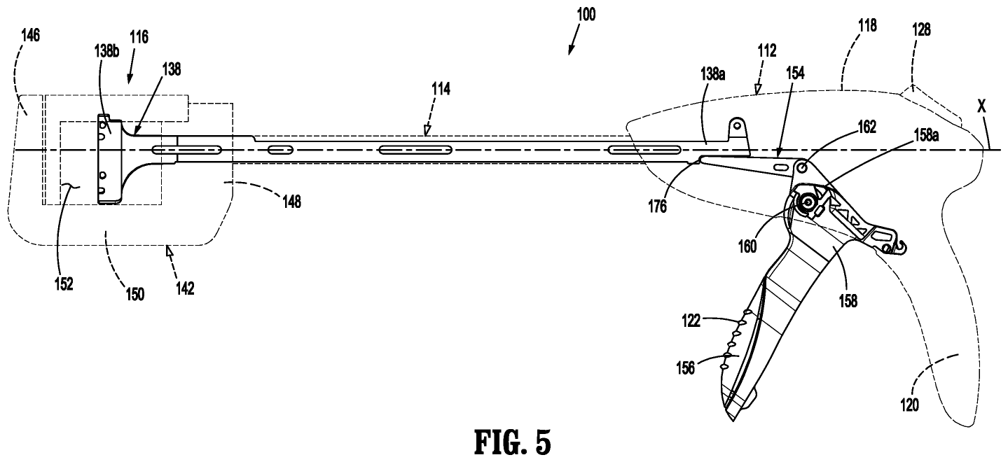

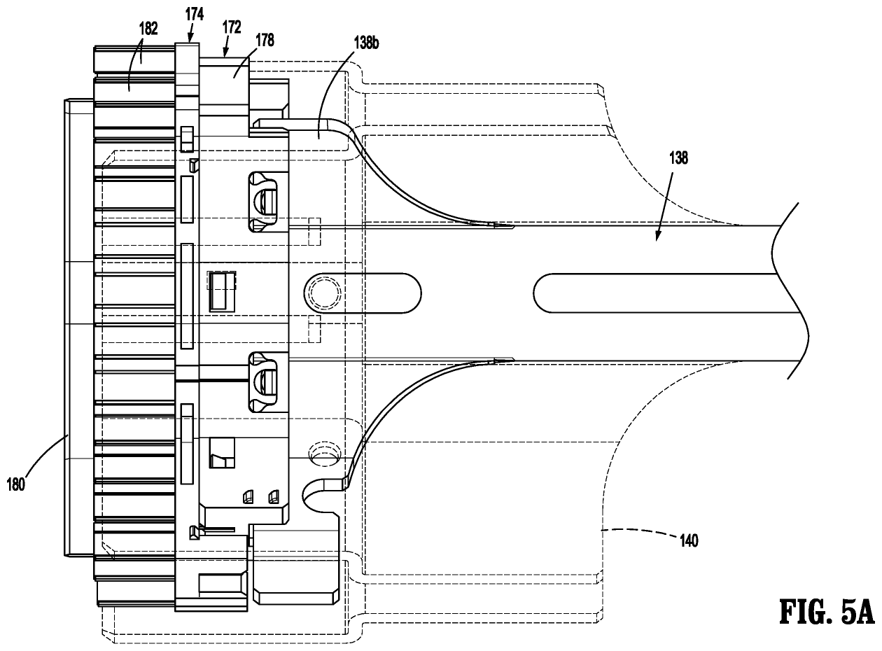

The present disclosure is directed to a stapling device shown generally as stapling device 100 in which the firing mechanism is modified to minimize the torque required to fire the stapling device 100, i.e., move the trigger 122 from an unactuated position (FIG. 5) towards an actuated position.

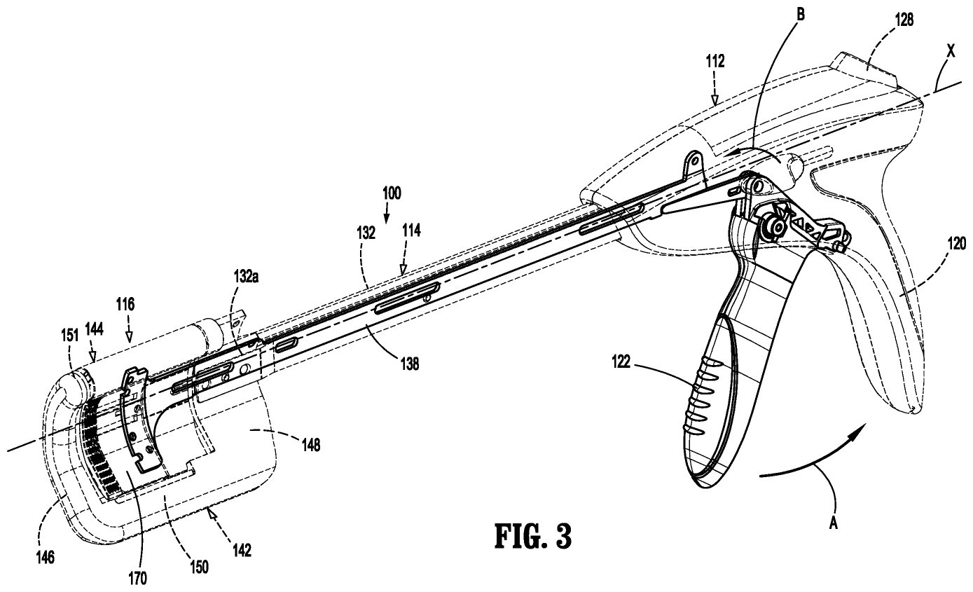

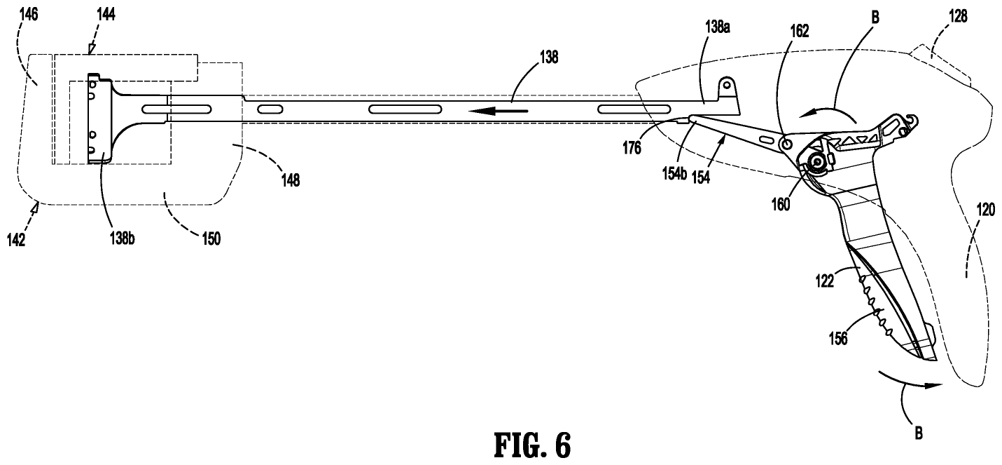

FIGS. 3-6 illustrate the stapling device 100 according to aspects of the disclosure. The stapling device 100 is similar to the stapling device 10 (FIG. 1) and includes a handle assembly 112, an elongate body 114 that extends distally from the handle assembly 112, and an end effector 116 that is supported on a distal portion of the elongate body 114. The elongate body 114 defines a longitudinal axis “X” (FIG. 5). The handle assembly 112 includes a housing 118 that defines a stationary handle 120 and supports a movable trigger 122. In aspects of the disclosure, the trigger 122 is supported by the housing 118 to pivot towards the stationary handle 120 in the direction of arrow “A” in FIG. 3 through a firing stroke from a non-actuated position (FIG. 3) to an actuated position (FIG. 6) to actuate the end effector 116, i.e., move the end effector 116 between open and clamped positions and fire the stapling device 100. The handle assembly 112 includes a release button 128 that can be depressed to move the end effector 116 from the clamped position to the open position. For a more detailed description of a representative handle assembly, see the '508 patent.

The stapling device 100 includes a frame 132 that extends distally from the handle assembly 112 and includes a distal portion 132a that supports the end effector 116. The frame 132 includes spaced frame members that define a channel that receives a thrust bar 138 and an alignment pin pusher (not shown). The thrust bar 138 is movable within the channel of the frame 132 between retracted and advanced positions in response to movement of the trigger 122 through the firing stroke to actuate the stapling device 100. The channel defined by the frame 132 also receives a clamp slide assembly 140 (FIG. 5A) that extends into the end effector 116 as described in further detail below. For a more detailed description of the interaction between the trigger 122, the thrust bar 138, and the clamp slide assembly 140, see the '508 patent.

The end effector 116 includes an end effector frame 142 and a cartridge assembly 144. The end effector frame 142 is secured to the distal portion of the frame 132 of the stapling device 100 and includes a first transverse portion 146, a second transverse portion 148, and a longitudinal portion 150 that interconnects the first transverse portion 146 and the second transverse portion 148. The first and second transverse portions 146, 148 are spaced from each other to define a gap 152 (FIG. 5) between the first and second transverse portions 146, 148. In some aspects of the disclosure, the first and second transverse portions 146, 148 are curved along axes transverse to the longitudinal axis “X” of the elongate body 114 of the stapling device 100. In some aspects of the disclosure, the first and second transverse portions 146, 148 are formed from a plurality of linear portions, e.g., three, that are positioned at angles in relation to each other to define a curved configuration. The first transverse portion 146 of the end effector frame 142 supports an anvil assembly 151.

FIG. 4 illustrates a firing mechanism of the stapling device 100 which includes the thrust bar 138, the trigger 122, and a firing link 154. In aspects of the disclosure, the trigger 122 includes a first end portion 156 that forms a grip and a second end portion 158. The second end portion 158 of the trigger 122 is pivotably secured to the housing 118 (FIG. 3) by a first pivot member 160 and pivotably secured to the firing link 154 by a second pivot member 162. In aspects of the disclosure, the first pivot member 160 in integrally formed with the trigger 122. The second pivot member 162 is positioned nearer a second end 158a of the trigger 122 than the first pivot member 160 such that when the trigger 122 is moved through the firing stroke from the unactuated position (FIG. 3) towards the actuated position (FIG. 6), the second pivot member 162 moves distally in the direction indicated by arrow “B” in FIG. 6 about the first pivot member 160.

In aspects of the disclosure, the firing link 154 has a first end 154a that defines a C-shaped clip and the second end portion 158 of the trigger 122 defines a clevis 164. The second pivot member 162 is supported within the clevis 164 and the first end 154a of the firing link 154 is clipped about the second pivot member 162 to pivotably secure the firing link 154 to the second end portion 158 of the trigger 122.

FIG. 4A illustrates the firing mechanism of the stapling device 100. As illustrated, the firing link 154 defines a longitudinal axis “Y” that extends through the second pivot member 162 and has a distal portion 154b. The proximal portion 154a of the firing link is pivotably secured to the second end portion 158 of the trigger 122 by the second pivot member 162. The distal portion 154b of the firing link 154 has a tapered configuration that is received in a recess 176 formed in the proximal end portion 138a of the thrust bar 138. When the firing mechanism of the stapling device 100 is assembled, an axis “Z” extends through the first and second pivot members 160, 162, and the thrust bar 138 defines a longitudinal axis “X1” that is substantially parallel to the longitudinal axis “X” of the elongate body 114. As illustrated, the first pivot member 160 is positioned proximally of the second pivot member 162. The axes “X” and “Y” define an angle define an angle Q and the axes “Y” and “Z” define an angle β.

The firing mechanism of the stapling device 100 is configured such that the angle β is larger in the unactuated position than in the prior art such that the mechanical advantage is greatest at the end of the firing stroke where load is at its highest. In aspects of the disclosure, angle β can be from about 95 degrees to about 110 degrees in the unactuated position of the trigger 122, and from about 155 degrees to about 170 degrees in the actuated position of the trigger 122. In the unactuated position, Ω is from about 9 degrees to about 13 degrees when the trigger 122 is in the unactuated position and from about 7 degrees to about 11 degrees when the trigger 122 is in the actuated position. In some aspects of the disclosure, Ω is greater in the unactuated position than it is in the actuated position.

FIG. 5A illustrates a distal portion of the stapling device 10 including the end effector 116, the clamp slide assembly 140, and portions of the cartridge assembly 144 (FIG. 3). The cartridge assembly 144 is supported within a distal portion of the clamp slide assembly 140. The clamp slide assembly 140 is movable within the gap 152 to move the cartridge assembly 144 in relation to the anvil assembly 151 (FIG. 3) between spaced and clamped positions. The cartridge assembly 144 includes a cartridge body 170 (FIG. 3), a knife assembly 172 and a pusher 174. The cartridge body 170 defines a cavity that receives the knife assembly 172 and the pusher 174 such that the knife assembly 172 and the pusher 174 are movable within the cartridge body 170 (FIG. 3) between retracted and advanced positions. The knife assembly 172 includes a knife holder 178 and a cutting blade 180 that is secured to and extends distally from the knife holder 178. The pusher 174 is positioned within the cartridge body 170 (FIG. 3) distally of the knife holder 178 and defines a knife slot (not shown) that receives the cutting blade 180. The pusher 174 includes fingers 182 that engage staples (not shown) supported within staple receiving slots of the cartridge body 170.

The distal portion of the thrust bar 138 includes a widened portion 138b that is received in the end effector 116 and is engaged with the knife holder 178 of the knife assembly 172. When the thrust bar 138 is advanced via movement of the trigger 122 through the firing stroke, the knife assembly 172 is moved within the cartridge body 170 (FIG. 3) from its retracted position towards its advanced position to advance the cutting blade 180 from a position recesses within the cartridge body 170 to a position extending from the cartridge body 170. The knife holder 178 is engaged with the pusher 174 such that movement of the knife assembly 172 from its retracted position towards its advanced position moves the pusher 174 within the cartridge body 170 (FIG. 3) from its retracted position towards it advanced position to eject staples from the cartridge body 170 (FIG. 3 into the anvil assembly 151.

FIG. 6 illustrates the stapling device 100 as the stapling device 100 is fired, i.e., as the trigger 122 in moved through the firing stroke from its unactuated position to its actuated position. When the trigger 122 is moved through its firing stroke in the direction of arrow “A”, the second pivot member 162 is rotated in a distal direction about the first pivot member 160 to advance the firing link 154 distally. As described above, the distal portion 154b of the firing link 154 is received in the recess 176 in the proximal end portion 138a of the thrust bar 138 such that the advancement of the firing link 154 causes longitudinal advancement of the thrust bar 138. Advancement of the thrust bar 138 causes movement of the knife assembly 172 (FIG. 5A) and the pusher 174 within the cartridge body 170 (FIG. 3).

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A surgical stapling device comprising:
a handle assembly including a housing, a firing link, and a trigger, the housing defining a stationary handle, the trigger mounted to the housing by a first pivot member and having a first portion defining a grip and a second portion having a second end spaced from the first portion, the firing link defining a longitudinal axis “Y” and having a first end portion and a second end portion, the first end portion of the firing link coupled to the second portion of the trigger by a second pivot member, the trigger being pivotable about the first pivot member towards the stationary handle through a firing stroke from an unactuated position to an actuated position, wherein an axis “Z” extends through the first and second pivot members;
an elongate body extending distally from the handle assembly, the elongate body including a frame and a thrust bar movably supported on the frame between retracted and advanced positions, the frame having a distal portion and a proximal portion, the thrust bar defining a longitudinal axis “X” and having a distal portion and a proximal portion, the proximal portion of the thrust bar engaged with the second end portion of the firing link such that movement of the trigger through the firing stroke moves the thrust bar from the retracted position to the advanced position; and
an end effector supported on the distal portion of the frame of the elongate body and including an end effector frame, an anvil assembly, and a cartridge assembly, the end effector frame supporting the anvil assembly and the cartridge assembly, the cartridge assembly including a cartridge body, a knife assembly, and a pusher, the knife assembly engaged with the distal portion of the thrust bar such that movement of the thrust bar from its retracted position to its advanced position moves the knife assembly and the pusher within the cartridge body from retracted positions to advanced positions;
wherein the axes “Y” and “Z” define an angle β, the angle β being from about 95 degrees to about 110 degrees when the trigger is in the unactuated position and from about 155 degrees to about 170 degrees when the trigger is in the actuated position.

2. The surgical stapling device of claim 1, wherein the axes “X” and “Y” define an angle Ω, and when the trigger is in the unactuated position, the angle α is greater than the angle α when the trigger is in the actuated position.

3. The surgical stapling device of claim 1, wherein the end effector frame includes first and second transverse portions and a longitudinal portion that interconnects the first and second transverse portions, the first and second transverse portions defining a gap.

4. The surgical stapling device of claim 3, wherein the anvil assembly is supported on the first transverse portion and the cartridge assembly is supported adjacent the second transverse portion.

5. The surgical stapling device of claim 4, further including a clamp slide assembly including a distal portion supported within the gap, the clamp slide assembly being movable between retracted and advanced positions to move the cartridge assembly in relation to the anvil assembly between open and clamped positions.

6. The surgical stapling device of claim 1, wherein the proximal portion of the thrust bar defines a recess and the distal portion of the firing link is received within the recess.

7. The surgical stapling device of claim 1, wherein the first end portion of the firing link includes a C-shaped clip that receives the second pivot member.

8. The surgical stapling device of claim 1, wherein the first pivot member is positioned between the second pivot member and the grip of the trigger.

9. The surgical stapling device of claim 1, wherein the first pivot member is positioned proximally of the second pivot member when the trigger is in the unactuated position.

10. A handle assembly comprising:
a housing, a firing link, a trigger, and a thrust bar, the housing defining a stationary handle, the trigger mounted to the housing by a first pivot member and having a first portion defining a grip and a second portion having a second end spaced from the first portion, the firing link defining a longitudinal axis “Y” and having a first end portion and a second end portion, the first end portion of the firing link coupled to the second portion of the trigger by a second pivot member, the trigger being pivotable about the first pivot member towards the stationary handle through a firing stroke from an unactuated position to an actuated position, the thrust bar extending from a distal portion of the housing and defining a longitudinal axis “X”, wherein an axis “Z” extends through the first and second pivot members, and wherein the axes “Y” and “Z” define an angle β, the angle β being from about 95 degrees to about 110 degrees when the trigger is in the unactuated position and from about 155 degrees to about 170 degrees when the trigger is in the actuated position.

11. The handle assembly of claim 10, wherein the axes “X” and “Y” define an angle α, and the angle α, when the trigger is in the unactuated position, is greater than the angle α when the trigger is in the actuated position.

12. The handle assembly of claim 10, wherein the proximal portion of the thrust bar defines a recess and the distal portion of the firing link is received within the recess.

13. The handle assembly of claim 10, wherein the first end portion of the firing link includes a C-shaped clip that receives the second pivot member.

14. The handle assembly of claim 10, wherein the first pivot member is positioned between the second pivot member and the grip of the trigger.

15. The handle assembly of claim 10, wherein the first pivot member is positioned proximally of the second pivot member when the trigger is in the unactuated position.

16. A surgical stapling device comprising:
a handle assembly including a housing, a firing link, and a trigger, the housing defining a stationary handle, the trigger mounted to the housing by a first pivot member and having a first portion defining a grip and a second portion having a second end spaced from the first portion, the firing link defining a longitudinal axis “Y” and having a first end portion and a second end portion, the first end portion of the firing link coupled to the second portion of the trigger by a second pivot member, the trigger being pivotable about the first pivot member towards the stationary handle through a firing stroke from an unactuated position to an actuated position, wherein an axis “Z” extends through the first and second pivot members;
an elongate body extending distally from the handle assembly, the elongate body including a frame and a thrust bar movably supported on the frame between retracted and advanced positions, the frame having a distal portion and a proximal portion, the thrust bar defining a longitudinal axis “X” and having a distal portion and a proximal portion, the proximal portion of the thrust bar engaged with the second end portion of the firing link such that movement of the trigger through the firing stroke moves the thrust bar from the retracted position to the advanced position; and
an end effector supported on the distal portion of the frame of the elongate body and including an end effector frame, an anvil assembly, and a cartridge assembly, the end effector frame supporting the anvil assembly and the cartridge assembly, the cartridge assembly including a cartridge body, a knife assembly, and a pusher, the knife assembly engaged with the distal portion of the thrust bar such that movement of the thrust bar from its retracted position to its advanced position moves the knife assembly and the pusher within the cartridge body from retracted positions to advanced positions;
wherein the axes “X” and “Y” define an angle α, the angle α, when the trigger is in the unactuated position, being greater the angle α when the trigger is in the actuated position.

17. The surgical stapling device of claim 16, wherein the axes “Y” and “Z” define an angle β, the angle β being from about 95 degrees to about 110 degrees when the trigger is in the unactuated position and from about 155 degrees to about 170 degrees when the trigger is in the actuated position.

18. The surgical stapling device of claim 16, wherein the proximal portion of the thrust bar defines a recess and the distal portion of the firing link is received within the recess.

19. The surgical stapling device of claim 16, wherein the first pivot member is positioned between the second pivot member and the grip of the trigger.

20. The surgical stapling device of claim 16, wherein the first pivot member is positioned proximally of the second pivot member when the trigger is in the unactuated position.
